(19) United States
(12) Patent Application Publication
(10) Pub. No.: US 2009/0186389 A1
(43) Pub. Date: Jul. 23, 2009

(54) MICROORGANISM WHOSE ACTIVITY OF ASPARTATE SEMIALDEHYDE DEHYDROGENASE IS ENHANCED AND THE PROCESS FOR PRODUCING L-THREONINE USING THE MICROORGANISM

(75) Inventors: Jong-il Choi, Seoul (KR); Young-lyeol Yang, Kyeonggi-do (KR); Young-hoon Park, Kyeonggi-do (KR)

Correspondence Address: SWANSON & BRATSCHUN, L.L.C., 8210 SOUTHPARK TERRACE, LITTLETON, CO, 80120, US

(73) Assignee: CJ CHEILJEDANG CORPORATION, Seoul (KR)

(21) Appl. No.: 12/375,119

(22) PCT Filed: Aug. 3, 2007
(86) PCT No.: PCT/KR2007/003746

§ 371 Date: Feb. 23, 2009

(30) Foreign Application Priority Data
Aug. 10, 2006 (KR) 10-2006-0075814

(51) Int. Cl. C12P 13/08; C12N 1/21

(52) U.S. Cl. 435/115; 435/252.33

(57) ABSTRACT

The present invention relates to a microorganism producing L-threonine with increased L-threonine production efficiency by the increased activity of aspartate semialdehyde dehydrogenase in L-threonine biosynthesis pathway,

### TECHNICAL FIELD

The present invention relates to identifying the unknown function of usg gene of E. coli using bioinformatics and a microorganism producing L-threonine that the expression of usg gene is increased and a method for producing L-threonine using the microorganism. More particularly, the present invention a microorganism producing L-threonine with high yield by increasing the expression of usg gene based on the result that the nucleotide sequence of usg gene of E. coli, whose functions had been unknown so far, has significant homology with the amino acid sequence of aspartate semialdehyde dehydrogenase involved in the L-threonine biosynthesis pathway by performing the similarity search in the nucleotide sequence of usg gene of E. coli using a bioinformatics and a method for producing L-threonine using the same.

### BACKGROUND ART

L-threonine is one of the essential amino acids, which has been widely used as an additive for feeds and foods as well as a synthetic raw material for medicinal supplies including injectable solutions and other medical drugs. L-threonine is produced mainly by microorganism fermentation for which artificial mutants induced from wild type microorganisms of Escherichia coli, Corynebacterium sp., Serratia sp. or Providencia sp. are used as a producing strain. Japanese Laid-Open Patent Publication No. Hei 2-219582 describes a method using microorganism of the genus of Providentia which is resistant to α-amino-β-hydroxy valeric acid, L-methionine, thiaisoleucine, oxythiamine and sulfaguanidine and has a requirement for L-leucine and a leaky requirement for L-isoleucine. Korean Patent No. 58286 describes a microorganism of the genus of Escherichia coli which is capable of producing L-threonine and is resistant to L-methionine analogues, L-threonine analogues, L-lysine analogs and α-amino butyric acid and has a requirement for methionine and a reaky requirement for isoleucine.

The methods of the prior art selects a microorganism with effective mutation by randomly transforming them. However, where the method of microorganism development by such artificial mutation is used, the characteristics of the mutated microorganism cannot be precisely determined and moreover the mutated microorganism might have a mutation that inhibits the growth of the microorganism. Therefore, it is required to develop a novel method for generating a microorganism capable of control more precisely which can improve or inhibit only desired characteristics.

Recently, the sequence of whole genome of E. coli etc. is identified and the data of various bioinformatics is accumulated. Therefore, studies on the functions of unknown genes have been accelerated.

Based on the advance of techniques enabling sequence analysis of the whole genome of E. coli and the accumulation of wide information by bio-informatics, studies on the functions of unknown genes have been accelerated.

Although the sequences of the whole genomes of many bacteria such as E. coli have been identified, some of their functions still remain unknown. The nucleotide sequence and amino acid sequence are closely related with the functions or structures of their target proteins. Therefore, investigation of homology between already identified sequences can be a help for the studies on the functions of proteins.

Thus, the present inventors studied to increase production efficiency of L-threonine using a microorganism. As a result, the inventors have confirmed that the protein encoded by the E. coli gene usg (NCBI GI: 16130524: SEQ. ID. NO: 5), whose sequence has been identified but whose functions have not been identified yet in E. coli has significant similarity with the amino acid sequence of aspartate semialdehyde dehydrogenase. And the inventors have further completed this invention that the L-threonine production can be increased by increasing the expression of usg based on the above.

### DISCLOSURE OF INVENTION

### Technical Problem

It is an object of the present invention to provide a recombinant microorganism with increased L-threonine production by increasing the expression of usg gene of E. coli.

It is also an object of the present invention to provide a method for producing L-threonine by culturing the recombinant microorganism having the improved L-threonine producing capacity.

### Technical Solution

The above objects of the present invention can be achieved by the following embodiments of the present invention.

The present invention is described in detail hereinafter.

To achieve the above objects, the present invention provides a microorganism producing L-threonine with increased L-threonine production efficiency by the increased activity of aspartate semialdehyde dehydrogenase in L-threonine biosynthesis pathway.

In a preferred embodiment of the present invention, the aspartate semialdehyde dehydrogenase may be encoded by usg gene derived from E. coli.

In a preferred embodiment of the present invention, a microorganism that has L-threonine production capacity can be the one transformed with the recombinant vector containing usg gene.

The microorganism producing L-threonine of the present invention can be any microorganism that is able to produce L-threonine including Escherichia coli, Corynebacterium sp., Serratia sp. and Providencia sp. bacteria, and among these E. coli is preferred. More preferably, Escherichia coli TF5015 (Global Analysis of Transcriptomes and Proteomes of a Parent Strain and an LThreonine-Overproducing Mutant Strain, Jin-Ho Lee, Dong-Eun Lee, Bheong-Uk Lee, and Hak-Sung Kim, JOURNAL OF BACTERIOLOGY, September 2003, p. 5442-5451) having a requirement for methionine and a leaky requirement for isoleucine and at the same time is resistant to α-amino-β-hydroxyvaleric acid, 2-aminoethyl-L-cysteine and L-azetidine-2-carboxylic acid can be used.

The gene usg has been located on the genome of E. coli, whose sequence has been identified but not the functions (NCBI GI: 16130254). In the present invention, the sequence of usg was compared with those of enzymes involved in L-threonine biosynthesis by bioinformatics technique. As a result, the gene was identified to have significant similarity with the amino acid sequence of aspartate semialdehyde dehydrogenase. Aspartate semialdehyde dehydrogenase seems to be involved in the ratelimiting step of L-threonine biosynthesis pathway in E. coli (FIG. 2). Therefore, the increase of the expression of usg was expected to increase the production capacity of L-threonine.

In a preferred embodiment of the present invention, to increase the expression of a target gene, the method increasing the expression of gene by introducing the host cell using a multicopy number vector is used. The vector, at this time, can be a wild-type one or a recombinant plasmid, cosmid, virus or bacteriophage. The vector is generally exemplified by natural or recombinant plasmid, cosmid, virus and bacteriophage. Preferably, low copy number pCL1920 plasmid vector which is spontaneously multipliable in Escherichia sp. bacteria can be used.

Also, the recombinant vector of the present invention can be prepared by the conventional method known to those in the art. For example, it is prepared by ligation of a gene identified the function by bioinformatics analysis to a proper vector containing a promoter and a terminator for the expression by using such restriction enzymes as EcoRV and HindIII. The promoter for expression can be trc, tac, lac, and a promoter of E. coli aroF gene. A terminator can be used for the effective expression.

In a preferred embodiment of the invention, the microorganism producing L-threonine which was transformed with the recombinant vector could be E. coli TF64212 (Accession No: KCCM-10768).

Particularly, the transformed cells of the present invention can be prepared by transforming host cells with the above recombinant vector by the conventional method. The host cells are L-threonine producing microorganism, preferably belongs to Gram-negative bacteria and more preferably belongs to Escherichia sp. In a preferred embodiment of the invention, E. coli TF5015 (Global Analyses of Transcriptomes and Proteomes of a Parent Strain and an L-Threonine-Overproducing Mutant Strain, Jin-Ho Lee, Dong-Eun Lee, Bheong-Uk Lee, and Hak-Sung Kim, JOURNAL OF BACTERIOLOGY, September 2003, p. 5442-5451) was transformed with the above recombinant vector (pCL-P_{aroF}-usg) to construct E. coli TF64212. The E. coli TF64212 was aroF deposited at KCCM (Korean Culture Center of Microorganism, Eulim Buld., Hongje-1-Dong, Seodaemun-Ku, Seoul, 361-221, Korea) on Jul. 24, 2006 (Accession No: KCCM-10768).

The recombinant microorganism for the production of L-threonine can produce L-threonine with high yield than in the microorganism before transformation by increasing the expression of usg identified to have the activity of aspartate semialdehyde dehydrogenase by bioinformatics.

In another preferred embodiment of the invention, the present invention provides a method for producing L-threonine from the recombinant microorganism for the production of L-threonine with increased production efficiency resulted from the increased aspartate semialdehyde dehydrogenase activity. The processes for the culture of a microorganism to mass-produce L-threonine in a recombinant microorganism and for separation of L-threonine from the culture are well informed to those in the art.

### BRIEF DESCRIPTION OF THE DRAWINGS

The application of the preferred embodiments of the present invention is best understood with reference to the accompanying drawings, wherein:

FIG. 1 is a diagram illustrating the construction procedure of the recombinant vector pCL-P_{aroF}-usg.

FIG. 2 is a diagram illustrating the L-threonine biosynthesis pathway.

### BEST MODE FOR CARRYING OUT THE INVENTION

Practical and presently preferred embodiments of the present invention are illustrative as shown in the following Examples. However, the following examples are provided for illustrative purposes only and are not intended to limit the scope of the invention.

### Example 1

### Identifying the Function of Use Gene of E. coli Using Bioinformatics

Since the whole genome sequence of E. coli was identified, various bioinformatic techniques have been developed to predict the unknown functions of hypothetical proteins. Bioinformatic analysis used herein is a technique to predict the functions of the gene using the nucleotide sequence of a gene or amino acid sequence of a protein resulted from the transcription and translation of a gene.

In this example, an enzyme with known function which have sequence similarity was screened by BLAST search as bioinformatics using the nucleotide sequence of usg gene with unknown functions in E. coli. As a result, USG protein encoded by usg gene was identified to have 28% of similarity in amino acid sequence with aspartate semialdehyde dehydrogenase.

```
>ref|NP_417891.1| aspartate-semialdehyde dehydrogenase; aspartate-semialdehyde
                    dehydrogenase, NAD(P)-binding [Escherichia coli K12]
                    Length = 367
  Score = 29.3 bits (64), Expect = 0.39
  Identities = 18/63 (28%), Positives = 30/63 (47%), Gaps = 1/63 (1%)
Query = 6 NIAVLGATGAVGEALLETLAE-RQFPVGEIYALARNESAGEQLRFGGKTITVQDAAEFDW 64
                      N+ +G G VG L++ + E R F + ++ FGG T T+QDA + +
Sbjct = 3 NVGFIGWAGMVGSVLMQRMVEERDFDAIPPYFFSTSQLGQAAPSFGGTTGTLQDAFDLEA 62
Query = 65 NQA 67
                        +A
Sbjct = 63 LKA 65
```

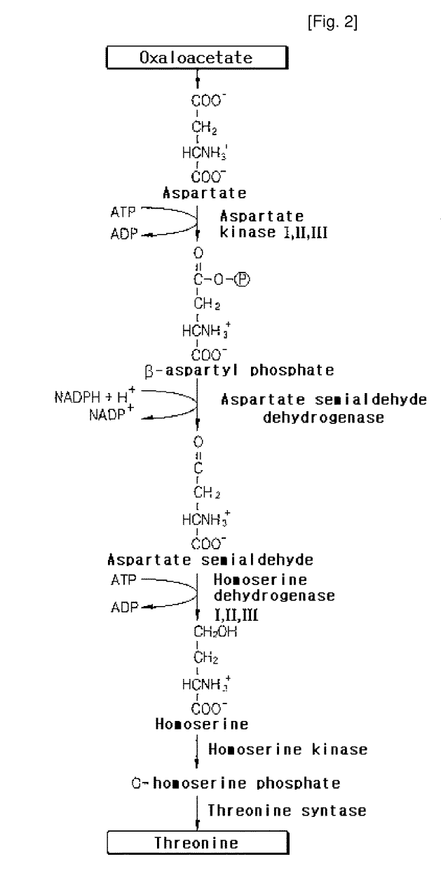

Aspartate semialdehyde dehydrogenase having a significant similarity with usg is an enzyme that converts aspartate semialdehyde into L-aspartyl-4-phosphate in L-threonine biosynthesis as shown in FIG. 2. Rate-limiting step in L-threonine biosynthesis pathway of E. coli is also catalyzed by the aspartate semialdehyde dehydrogenase. Therefore, the increase of the expression of usg was expected to increase the production capacity of L-threonine.

### Example 2

### Preparation of a Recombinant Vector Containing usg Gene

To identify the improvement of L-threonine production capacity by the increase of the expression of usg whose functions have been predicted by bioinformatics, usg gene was over-expressed using a plasmid having multicopy number in E. coli.

First, promoter necessary for the expression was inserted into a plasmid vector pCL1920. To use the promoter of aroF gene existing in the chromosome of E. coli, primers 1 (SEQ. ID. NO: 1) and 2 (SEQ. ID. NO: 2) of Table 1 were prepared, respectively. Approximately 700 base-pairs of aroF gene promoter region were amplified by PCR [Sambrook et al, Molecular Cloning, a Laboratory Manual (1989), Cold Spring Harbor Laboratories] (PCR condition: Denaturing=95° C., 30 sec/Annealing=53° C., 30 sec/Polymerization=72° C., 1 min, 30 cycles) using the chromosomal DNA of wild-type E. coli W3110 as a template. The obtained DNA fragment was digested with KpnI and EcoRV, followed by ligation with pCL-plasmid digested with the same restriction enzymes using T4 DNA ligase to prepare pCL-P_{aroF}.

TABLE 1
Sequences of primers 1 and 2

| | | |
|---|---|---|
| Primer 1 | 5′-cgg ggt acc tgc tgg tca agg ttg gcg cgt-3′ | (SEQ. ID. NO: 1) |
| Primer 2 | 5′-ccg gat atc gat cct gtt tat gct cgt ttg-3′ | (SEQ. ID. NO: 2) |

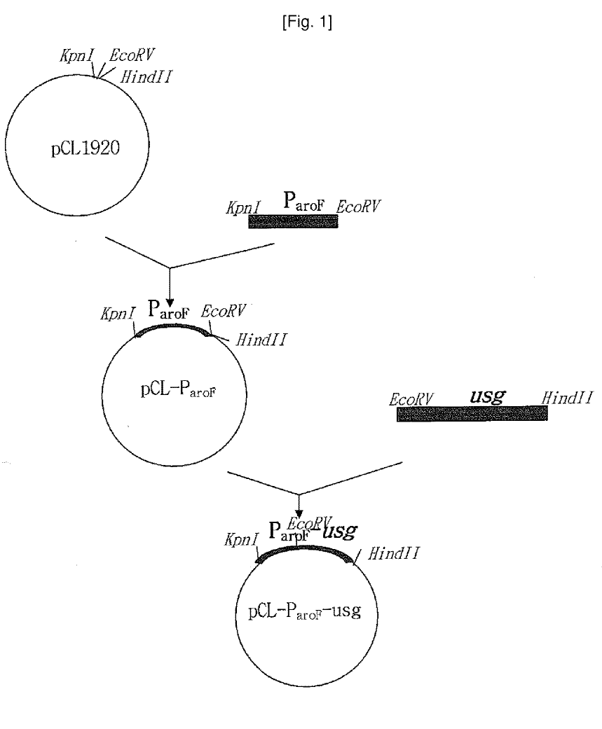

Also, primers having SEQ. ID. NO: 3 and NO:4 of table 2, respectively, were prepared for the cloning of usg gene from the wild-type E. coli W3110. The nucleotide sequence of usg (NCBI GI: 1613054: SEQ. ID. NO: 5) has already been reported. Approximately 1,014 base-pairs of usg gene were amplified by PCR [Sambrook et al, Molecular Cloning, a Laboratory Manual (1989), Cold Spring Harbor Laboratories] (PCR condition: Denaturing=95° C., 30 sec/Annealing=53° C., 30 sec/Polymerization=72° C., 1 min, 30 cycles) using the chromosomal DNA of wild-type E. coli W3110 as a template. The obtained DNA fragment was digested with the restriction enzymes EcoRV and HindIII, followed by ligation with pCL-P_{aroF} digested with the same enzymes using T4 DNA ligase, resulting in the preparation of the recombinant vector pCLParoF-usg as shown in FIG. 1.

TABLE 2
Sequences of primers 3 and 4

| | | |
|---|---|---|
| Primer 3 | 5′-ggg gat atc atg tct gaa ggc tgg aac-3′ | (SEQ. ID. NO: 3) |
| Primer 4 | 5′-ggg aag ctt tta gta cag ata ctc ctg-3′ | (SEQ. ID. NO: 4) |

### Example 3

### Comparison with L-threonine Production Capacity of a Recombinant Microorganism Over-Expressing usg Gene

In this example, E. coli TF5015 producing L-threonine was transformed with the recombinant vector (pCL-ParoF-usg) prepared in Example 2. The obtained transformant TF64212 (KCCM-10768) was cultured in a flask titer medium having the composition as described in Table 3. Centrifugation was performed to separate supernatant from the culture solution and the supernatant proceeded to liquid chromatography to measure the concentration of threonine. The concentration of L-threonine were compared between E. coli TF5015 and the transformant TF64212. Mean value of the results from 3 flasks was used as the concentration of L-threonine.

TABLE 3
Flask titer medium

| Composition | Concentration (g/L) |
|---|---|
| Glucose | 70 |
| Yeast extract | 2 |
| Ammonium sulfate | 28 |
| Magnesium sulfate | 0.5 |
| Ferrous sulfate | 0.005 |
| Manganese sulfate | 0.005 |
| L-methionine | 0.15 |
| Calcium carbonate | 30 |
| Potassium dihydrogenphosphate | 1 |

As a result, as shown in Table 4, the concentration of L-threonine in the transformant TF64212 (KCCM-10768) increased by 20.8% in comparison to the E. coli TF5015.

TABLE 4
L-threonine concentration

| Strain | L-threonine (g/L) |
|---|---|
| TF5015 | 9.6 |
| TF64212 | 11.6 |

### INDUSTRIAL APPLICABILITY

As explained hereinbefore, the unknown functions of usg gene in E. coli were predicted by bioinformatics, and as a result usg gene was identified to have significant similarity on amino acid sequence with aspartate semialdehyde dehydrogenase involved in L-threonine biosynthesis. Therefore, the increase of the expression of usg was expected to increase the production capacity of L-threonine. Particularly, the E. coli transformant TF64212 over-expressing usg was prepared from E. coli TF5015 producing L-threonine. And L-threonine production was increased by the culture of the transformant.

1. A microorganism producing L-threonine with increased L-threonine production efficiency by the increased activity of aspartate semialdehyde dehydrogenase in L-threonine biosynthesis pathway.

2. The microorganism according to claim 1, wherein the microorganism has a requirement for methionine and a reaky requirement for isoleucine and is resistant to α-amino-β-hydroxy valeric acid, 2-aminoethyl-L-cysteine and L-azetidine-2-carboxylic acid.

3. The microorganism according to claim 1, wherein the aspartate semialdehyde dehydrogenase is encoded by the usg gene (SEQ. ID. NO: 5).

4. The microorganism according to claim 3, wherein the microorganism is transformed with the recombinant vector containing usg coding the aspartate semialdehyde dehydrogenase.

5. The microorganism according to claim 4, wherein the transformed microorganism producing L-threonine is E. coli TF64212 (Accession No: KCCM-10768).

6. A method for producing L-threonine containing the step of the culture of the microorganism of claim 1.


SEQUENCE LISTING (5 sequences)

SEQ ID NO: 1 - DNA, 30 bp, Artificial Sequence
  primer 1
cggggtacctgctggtcaaggttggcgcgt

SEQ ID NO: 2 - DNA, 30 bp, Artificial Sequence
  primer 2
ccggatatcgatcctgtttatgctcgtttg

SEQ ID NO: 3 - DNA, 27 bp, Artificial Sequence
  primer 3 for usg
ggggatatcatgtctgaaggctggaac

SEQ ID NO: 4 - DNA, 27 bp, Artificial Sequence
  primer 4 for usg
gggaagcttttagtacagatactcctg

SEQ ID NO: 5 - DNA, 1014 bp, Escherichia coli
atgtctgaaggctggaacattgccgtcctgggcgcaactggcgctgtgggcgaagccctgcttgaaacgctggctgaacgtcagttcccggttggggaaatttatgcactggcacgtaacgaaagcgcaggcgaacaactgcgctttggtggtaagacaatcaccgtgcaggatgccgctgaattcgactggacgcaggcgcagctggcattttttgtcgcaggcaaagaagctaccgctgcctgggttgaagaagcgaccaactcaggttgcctggtgatcgacagcagtggattgtttgctctcgaacccgacgtaccgctggtggtgccggaagtaaacccgtttgtactgacagattaccggaaccggaatgtcatcgccgtaccagacagtctgaccagccagctgctggcggcactgaaaccgttaatcgatcagggcggtttatcacgtatcagcgttaccagcctgatttcagcctccgcccagggcaaaaaagcggtcgatgcgttagcggggcagagtgcgaaattgctcaacggcattccgattgacgaagaagatttcttcgggcgtcagctggcgttcaacatgctgccgttactgccggatagcgaaggtagcgtgcgtgaagaacgtcgtatcgttgacgaagtacgcaaaatcctgcaggacgaagggctgatgatttcggctagcgtcgtccaggcaccggtattctacggtcatgcccagatggtcaactttgaagctctgcgtccactggcagcagaagaagcgcgtgatgcgtttgttcaaggcgaagatattgtgctctctgaagagaacgaattcccaactcaggtaggtgatgcttcgggtacgccgcatctttctgttggctgcgtgcgtaatgactacggtatgccggagcaagtccagttctggtcggtggccgataacgttcgctttggcggcgcgctgatggcagtaaaaatcgccgagaaactggtgcaggagtatctgtactaa